(19) United States
(12) Patent Application Publication
(10) Pub. No.: US 2004/0023892 A1
(43) Pub. Date: Feb. 5, 2004

(54) Pharmaceutical composition based on a non-steroid anti-inflammatory agent

(76) Inventor: Veniamin A Khazanov, Tomsk (RU)

Correspondence Address: Llya Zborovsky, 6 Schoolhouse Way, Dix Hills, NY, 11746, US

(21) Appl. No.: 10/399,825

(22) PCT Filed: Apr. 12, 2001
(86) PCT No.: PCT/RU01/00132

(51) Int. Cl. A61K 31/70; A61K 31/60; A61K 31/415; A61K 31/19; A61K 35/78

(52) U.S. Cl. 514/23; 514/165; 514/406; 514/570; 424/774

(57) ABSTRACT

The invention relates to medicine. The inventive composition comprises a non-steroid anti-inflammatory agent and at least one compound potentiating a pharmaceutical action, decreasing the toxicity and side effects thereof. The amplifying composition is selected from diocarbonillic acids or salts thereof, amino acids or salts thereof, vitamins, poliphenolic compounds or products of animal and/or vegetable origin, containing the compound. The inventive composition comprises also a special additive agent representing a carbohydrate and/or sorbing agent. The inventive composition is characterized by a better expressed pharmacological action and low toxicity even during a long period of use.

### FIELD OF THE INVENTION

The invention relates to medicine, in particular to a pharmacology, and deals with non-steroid, anti-inflammatory agents (NAIA).

### BACKGROUND ART

In medical practice pharmaceutical compositions are used with the use of NAIA of various chemical structures. In particular, NAIA include derivatives of salicylic acid (acetyl salicylic acid, diflunizal), derivatives of acetic acid (indometacyn, sulindac, tolmetim, diklofenac), derivatives of propanoic acid (ibuprofen, naproxen, cetoprofen, surgam), derivatives of antranilic acid (mefenamovic acid, voltaren), derivatives of nicotinic acid (niflumovic acid), pyrazolons (butadion, analgin, amidopirin), oxicams (piroxicam), etc. For pharmaceutical compositions based on NAIA, properties of NAIA are also characteristic, such as anti-inflammatory, analgetic, fever-reducing, and also the ability of braking of thrombocyte aggregation (to reduce coagulation of blood). Main negative actions of NAIA are ulcerous (ulcering of mucous of intestine) and a toxic action of preparations (Kharkevich D. A. “Pharmacology”, Moscow, 1999, P 461-473).

A pharmaceutical composition based on NIAA is known, which is widely used in a medical practice, and it is acetyl salicylic acid with ascorbic acid (aspirin-C), in which ascorbic acid enhances increase of resistance of organism and reduction of permeability of capilliaries (Mashkovsky M. D. “Medicines,” Kharkov, Torsing, 1998, V.1 p167).

A composition which is the closest to the claimed composition is a combination of acetyl salicylic acid with diammonium salt of succinic acid with the ratio of components, mass %:

Acetyl salicylic acid 70-80; succinic-acid ammonium 20-30 (U.S. Pat. No. 2,135,185 for invention “Anti-inflammatory Agent”, published in BI no. 24 of Aug. 27, 1999), but the ratio of components and the use of concrete means in this composition do not allow to increase pharmacological activity of NAIA and to reduce toxicity and side effects during the use of other NAIA, which require significantly lower doses during treatment, including in combination with other compositions, which allow to achieve the best therapeutical effect with the absence of side effects.

The main problem which is solved by the proposed invention is an increase of pharmacological activity of NAIA and their combinations with other biologically active compositions, and also elimination of side effects and reduction of toxic effects of NAIA.

This problem is solved in that, in a pharmaceutical composition which contains a non-steroid anti-inflammatory agent, and also a composition which increases its pharmacological action and reduces side effects and toxicity, as said composition a composition is used which contains at least one substance from the group: double base carboxylic acids or their salts, amino acids or their salts, poliphenolic compounds or products of vegetable and/or animal origin which contain them, substances which have vitamin action, and also at least one special additive agent representing a carbohydrate and/or sorbent.

As the non-steroid anti-inflammatory compound, the composition can include acetyl salicylic acid.

The pharmaceutical composition in a preferable variant contains succinic acid as the carbonic acid.

As the product of animal original which contains polyphenolic compounds, it is optimal to include extract of thick leaf badan and/or extract of malt bare.

Glucose can be used as the special additive in the pharmaceutical composition.

In an optimal variant of the embodiment of the invention the pharmaceutical composition contains in mass %:

| | |
|---|---|
| Acetyl salicylic acid | 25-80 |
| Succinate of Ammonium | 5-30 |
| Extract of Badan | 5-20 |
| Ascorbic acid | 5-20 |
| Glucose | the rest |

The pharmaceutical composition additionally can contain a pharmaceutically acceptable carrier which is suitable for oral administration.

It is preferable to form pharmaceutical composition as a pill or a capsule.

This combination of characteristic features obviously is not derivable for an expert in this technical field. Therefore the technical solution corresponds to criteria of invention “novelty, inventive level”. This technical solution can be used in a clinical practice as NAIA which has stronger pharmacological effect and lower side effects. Therefore, it corresponds to the criteria of invention “industrial utility”.

The proposed composition has a higher pharmacological activity, lower general toxicity and side effects when compared with analogs contained in the prior art, especially during long-term use of the preparation. It allows to obtain a positive effect which was not known before.

As NAIA various combinations of groups mentioned above can be utilized. As the agent which enhances pharmacological activity of NAIA and reduces side effects, in the composition can be introduced various pharmacologically acceptable carboxylic acids, such as for example succinic, fumaric, maleic, malic, their potassium, sodium, ammonium, for example succinate of ammonium and other salts, amino acids preferably glycin, taurin, glutaminic acids, their salts, substances which possess vitaminic action such as ascorbic acids, vitamins of group B, retinols and others, poliphenolic compositions of vegetable and/or animal origin obtained in a pure form, for example kvertetsin, baikalin, or in the form of extracts such as for example extract of five-leaves pustir, thick-leaf badan, rastorop spotted, malt bare. Agents which increase activity are introduced in effective quantities which are varied depending on their nature and purpose of the composition. In the composition as a special additive carbohydrates and/or sorbents are introduced. They can provide influence on bioaccessibility of components of combined preparation (change of disintegration of a pill, sorption of active substances), cause metabolic effect (carbohydrates) and also limit local actions of NAIA in a stomach-intestine system, so as to influence the main pharmacological effect-increase of anti-inflammatory action, reduction of toxicity and reduction of side effects. As carbohydrates various mono, di, polisaccarides can be used, for example, glucose, fructose, saccharose, lactose, stucciose, etc. As the sorbents, such substances can be used as for example activated carbon, silicagel, hydroxy apatite, hydro-oxide of aluminum, wheat bran, starch, mkz.

The above presented concrete substances are used for illustration of the invention, but they do not limit it.

In addition to above mentioned components, the compositions can include when necessary also other substances which are well known in pharmacology additional substances, which allow to select this or that preferable way of introduction of composition. They can be solvents, sliding substances, powdering substances, taste additives, film-forming and other components.

Compositions can be in different forms, preferably for per oral administration such as solutions, syrups, powders, pills, capsules.

### DESCRIPTION OF VARIANTS OF CARRYING OUT THE INVENTION

The claimed properties of the proposed composition were proven in tests on animals and in a clinical practice, which are presented in the following examples.

### EXAMPLE 1

Increase of Anti-Inflammatory Action of NAIA.

Actions were investigated on anti-inflammatory activity of NAIA of the following possible components of the composition:

A) Extracts with high content of polyphenolic compositions—shlemnik of baikal (ESB) and badan thick—leaf (EBT) polyfenolic composition of flavenoid structure-silibinin (SB), kvertsetin (KV),

B) carboxylic acids—malic (M), fumaric (FUM), succinic (S) and its ammonium salt (S-NH₄).

C) Amino acids—glutaminic (GLU), glycin (GLy) 16, tsistein (TIS), taurin (TAU).

D) Substances which have vitaminic action—vitamins C, B₆, E, lipoic acid (LA).

As NAIA compounds were used from all main groups of preparations-acetyl salicylic acid (ASA), indometatsin (IM), naproxen (NP), voltaren (VL), niflumic acid (NA), butadion (BN), piroxikam (PK). A single dose of NAIA varies from 10 mg, for example for pyroxikam to 500 mg, for example for aspirin, and depends from a type of preparation, gravity of sickness, age, weight of a patient.

Tests were performed on breedless white mice-males with mass 22-24 g. The investigated preparations were introduced to animals through a probe into stomach once a day during five days in form of suspension on 1% starch mucous or aqueous solution. For selection of a dose of the used preparation literature data were used, obtained on various animals, or in clinical investigations on humans with consideration of recommendations of Ministry of Health Methodology of calculation (Rules of Performing Preclinical Investigations of Pharmokinetics of Medications, Moscow, 1998, p.12-13). ASA was used in optimal anti-inflammatory dose for mice-200 mg/kg (Mashkovsky M. D. “Medications” Kharkov, Torsing, 1998, V.1 p165-167), and other NAIA were used with consideration of recalculation of therapeutic dose of animal in the following doses:

IM (10) (Mashkovsky M. D. “Medicines,” Kharkov, Torsing, 1998, V.1 p173, NP (100), (Mashkovsky M. D. “Medications,” Kharkov, Torsing, 1998, V.1 p174), (VL (10) (Mashkovsky M. D. “Mediccations,” Kharkov, Torsing, 1998, V.1 p172), NA (100) (Mashkovsky M. D. “Medications,” Kharkov, Torsing, 1998, V.1 p176), BN (60) (Mashkovsky M. D. “Medications,” Kharkov, Torsing, 1998, V.1 p169-171), PK (8) ((Mashkovsky M. D. “Medications,” Kharkov, Torsing, 1998, V.1 p174).

Compositions which increase activity of NAIA are introduced into effective quantities which are varied depending on their nature and purpose of the composition. For example, carboxylic acids are introduced into the content in the quantity of 5 mg up to 500 mg for a single dose. The quantity of amino acid can constitute from 0.1 mg for example for taurine, to 500 mg for example for glutamine acid. Vitamins are in the content in biologically active doses, including from 2 mg for example for riboflavin to 500 mg for example for ascorbic acid. Phenolic compositions as a rule are introduced in the content in the quantity of 2 mg for example for rutin, up to 300 mg for example of extract of baikal shlemnik.

In concrete tests illustrated by the examples, doses of carboxylic acids and their salts were: S—Na (100), S—NH₄ (100), M (100), (Therapeutic action of malic acid, edited by M. N. Kondrashova, Puschino 1976, p. 49-55, p. 77-79), FUM (100), (Ueda H. et al. Reduction of cisplatin toxicity and lethality by sodium malate in mice, Biol. Pharm. Bull, 1998, Jan, 21:1, 34-43).

Doses of amino acids were: GLU (100)(Mashkovsky M. D. “Medications,” Kharkov, Torsing, 1998, V.2 p128-129), GLy (100)(Mashkovsky M. D. “Medications,” Kharkov, Torsing, 1998, V.2 p131), TIS(50) (Mashkovsky M. D. “Medications,” Kharkov, Torsing, 1998, V.2 p131), TAU(50) (Mashkovsky M. D. “Medications,” Kharkov, Torsing, 1998, V.2 p132).

Doses of polyfenolic compositions or extracts which contain them were: ESB-150 mg/kg (Saifutdinov P. P. Influence of Shlemnik Baikal on energetic metabolism of brain of mice during hypoxy: Autoref. of dissertation for Candidate of Medical Science, Tomsk, 1997. 23p) EBT-100 mg/kg (Khazanov V. A., Smirnov N. B., Ilushenko S. V., Mitokhondrial effects in mechanism of cerebral protective action of extract of badan thick-leaf during hypoxy, bulletin of Experimental Biology and Medicine 2001, attachment 1, p. 34-38), kvertsetin-50 mg kg (Mashkovsky M. D. “Medications,” Kharkov, Torsing, 1998, V.2, p91), silibinin 50 mg/kg (Mashkovsky M. D. “Medications,” Kharkov, Torsing, 1998, V.1, p514-515).

Fractions of vitamins were: Vitamin C (50)(Mashkovsky M. D. “Medications,” Kharkov, Torsing, 1998, V.2, p88-89), Vitamin B₆ (10)(Mashkovsky M. D. “Medications,” Kharkov, Torsing, 1998, V.2, p80-81) Vitamin E (20) (Mashkovsky M. D. “Medications,” Kharkov, Torsing, 1998, V.2 p95-96), lipoic acid (20)(Mashkovsky M. D. “Medications,” Kharkov, Torsing, 1998, V.2, p104).

In one hour after the last introduction of preparations, mice were injected with 0.05 ml of 1% of solution of carragenin in isotonic solution of sodium chloride, under aneurism of right rear paw. After 3.5 hours, on a peak of inflammation, they were killed by dislocation of a neck area of spine. Swollen and healthy paws were compared, and a degree of suppression of swelling was determined in test groups when compared with a control group in accordance with the formula:

Increase of mass of paw in control-increase of mass of paw in test ______ 100 increase of mass of paw in control.

The results were processed statistically with the use of non-parametric criteria of Wilkoxon/Belenky M. L. “Elements of quantatative evaluation of pharmacological effect”, second edition, redone and added L, Medgiz, 1963, 152 p).

In table 2 results of separate action of investigated preparations were presented. It can be seen that all NAIA clearly limit development of inflammatory reaction, while other components of composition did not show pharmacological activity.

TABLE 1
Influence of investigated preparation on development of inflammatory
swelling of paw of mice after introduction of carragenin (n = 10)

| Group of Investigation | Increase of | Suppression of |
|---|---|---|
| (Dose | Mass of Limb, | Swelling, % to |
| mg/kg) | mg | Control |
| Control | 60.1 ± 7.4 | — |
| ASA 200 | 41.3 ± 5.2 | −31.3* |
| IM 10 | 19.0 ± 6.3 | −68.3* |
| NP 100 | 28.9 ± 5.4 | −51.9* |
| VL 10 | 24.3 ± 6.0 | −48.3* |
| NA 100 | 31.1 ± 6.0 | −48.3 |
| BN 60 | 32.4 ± 5.2 | −46.1 |
| PK 8 | 28.8 ± 4.9 | −52.0 |
| 5 100 | 59.6 ± 5.8 | −0.8 |
| S-NH4 100 | 63.5 ± 6.1 | +5.7 |
| SNa 100 | 61.7 ± 4.9 | +2.7 |
| APPL 100 | 58.4 ± 6.3 | −2.8 |
| FUM 100 | 59.3 ± 5.5 | −1.3 |
| GLU 100 | 62.3 ± 5.0 | +3.7 |
| GLy 100 | 63.0 ± 5.5 | +4.8 |
| TIS 50 | 61.5 ± 6.4 | +2.3 |
| TAU 50 | 60.0 ± 5.6 | −0.2 |
| ESB 150 | 59.4 ± 3.0 | −1.2 |
| EBT 100 | 60.5 ± 6.1 | −0.7 |
| KV 50 | 58.7 ± 3.6 | −2.3 |
| SB 50 | 62.3 ± 6.6 | +3.7 |
| Vit C 50 | 59.4 | −1.2 |
| Vit B₆ 10 | 60.5 | +0.7 |
| Vit E 20 | 58.7 | −2.3 |
| Lipoic acid LA 20 | 62.3 | +3.7 |

Note:
here and further (*) is a difference with a control value in its group).

TABLE 2
Influence of amino acids on anti-inflammatory activity NAIA during
development of swelling of paw of mice after introduction of carragenin
(N = 10).

| | | | | GLy | TAU |
|---|---|---|---|---|---|
| Preparation | Control | GLU (100) | | (50) | (50) |
| Control | 60.1 | 62.2 | 63.0 | 61.5 | 60.0 |
| ASA 200 | 41.3* | 38.6*^{Λ} | 37.7*^{Λ} | 31.6*^{Λ} | 37.0*^{Λ} |
| IM 10 | 19.0* | 15.4*^{Λ} | 16.0*^{Λ} | 15.9*^{Λ} | 15.2*^{Λ} |
| NP 100 | 28.9* | 25.1*^{Λ} | 26.0*^{Λ} | 22.7*^{Λ} | 23.8*^{Λ} |
| VL 10 | 24.3* | 20.2*^{Λ} | 21.1*^{Λ} | 19.5*^{Λ} | 21.0*^{Λ} |
| NA 100 | 31.1* | 26.8*^{Λ} | 26.2*^{Λ} | 24.9*^{Λ} | 26.2*^{Λ} |
| BN60 | 32.4* | 28.4*^{Λ} | 27.8*^{Λ} | 26.1*^{Λ} | 25.7*^{Λ} |
| PK 8 | 28.8* | 23.5*^{Λ} | 24.3*^{Λ} | 22.8*^{Λ} | 24.4*^{Λ} |

**Note:
Here and further (^{Λ}) is a difference with corresponding value in a column of control.

TABLE 3
Influence of polyphenolic compositions on anti-inflammatory activity of
NAIA during development of swelling of paw of mice after introduction of
carragenin (n = 10).

| | | | | KV | |
|---|---|---|---|---|---|
| Preparation | Control | ESB(100) | EBT(50) | (50) | SB(50) |
| Control | 60.1 | 59.4 | 60.5 | 58.7 | 62.3 |
| ASA 200 | 41.3* | 37.3*^{Λ} | 36.0*^{Λ} | 34.9*^{Λ} | 35.5*^{Λ} |
| IM 10 | 19.0* | 16.0*^{Λ} | 15.2*^{Λ} | 15.4*^{Λ} | 15.8*^{Λ} |
| NP 100 | 28.9* | 24.4*^{Λ} | 25.2*^{Λ} | 23.1*^{Λ} | 24.0*^{Λ} |
| VP. 10 | 24.3* | 19.5*^{Λ} | 20.7*^{Λ} | 19.0*^{Λ} | 19.3*^{Λ} |
| NA 100 | 31.1* | 25.7*^{Λ} | 25.2*^{Λ} | 24.0*^{Λ} | 25.9*^{Λ} |
| BN60 | 32.4* | 27.6*^{Λ} | 26.1*^{Λ} | 26.6*^{Λ} | 27.0*^{Λ} |
| PK 80 | 28.8* | 24.0*^{Λ} | 23.2*^{Λ} | 23.3*^{Λ} | 24.5*^{Λ} |

TABLE 4
Action of carcoxylic acids on anti-inflammatory activity of NAIA during
development of swelling of paw of mice after introduction of carragenine
(N = 10)

| | | AA-NH4 | AA-NA | APPL | FUM |
|---|---|---|---|---|---|
| Preparation | Control | (100) | (100) | (100) | (100) |
| Control | 60.1 | 63.5 | 61.7 | 58.7 | 59.3 |
| ASA 200 | 41.3* | 37.3*^{Λ} | 36.0*^{Λ} | 34.9*^{Λ} | 35.3*^{Λ} |
| IM 10 | 19.0* | 15.7*^{Λ} | 16.4*^{Λ} | 16.1*^{Λ} | 15.0*^{Λ} |
| NP 100 | 28.9* | 24.2*^{Λ} | 24.9*^{Λ} | 24.0*^{Λ} | 25.1*^{Λ} |
| VL. 10 | 24.3* | 19.9*^{Λ} | 20.0*^{Λ} | 19.2*^{Λ} | 19.5*^{Λ} |
| NA 100 | 31.1* | 26.8*^{Λ} | 26.0*^{Λ} | 25.5*^{Λ} | 26.7*^{Λ} |
| BN60 | 32.4* | 28.0*^{Λ} | 27.3*^{Λ} | 26.2*^{Λ} | 26.6*^{Λ} |
| PK 80 | 28.8* | 24.4*^{Λ} | 24.3*^{Λ} | 23.9*^{Λ} | 24.0*^{Λ} |

TABLE 5
Influence of vitamin preparations on anti-inflammatory activity NAIA
during
development of swelling of paw of mice after introduction of carragenin
(N = 10)

| | | | VIT B₆ | VIT E | LK |
|---|---|---|---|---|---|
| Preparation | Control | VIT C(50) | (10)(50) | (20) | (20) |
| Control | 60.1 | 59.4 | 60.5 | 58.7 | 62.3 |
| ASA 200 | 41.3* | 38.0*^{Λ} | 37.2*^{Λ} | 35.5*^{Λ} | 36.2*^{Λ} |
| IM 10 | 19.0* | 16.6*^{Λ} | 16.0*^{Λ} | 15.0*^{Λ} | 15.3*^{Λ} |
| NP 100 | 28.9* | 25.2*^{Λ} | 25.6*^{Λ} | 24.4*^{Λ} | 25.1*^{Λ} |
| VI. 10 | 24.3* | 20.9*^{Λ} | 2O.2*^{Λ} | 19.6*^{Λ} | 19.2*^{Λ} |
| NA 100 | 31.1* | 26.6*^{Λ} | 25.9*^{Λ} | 24.8*^{Λ} | 26.0*^{Λ} |
| BN60 | 32.4* | 28.1*^{Λ} | 27.3*^{Λ} | 26.8*^{Λ} | 27.2*^{Λ} |
| PK 80 | 28.8* | 24.4*^{Λ} | 23.8*^{Λ} | 24.0*^{Λ} | 23.3*^{Λ} |

TABLE 6
Action of polyphenolic compounds and vegetable extracts which contain
them on anti-inflammatory activity of combination NAIA with carboxylic
acids, amino acids, and vitamins during development of swelling of paw
of mice after introduction of carragenin (N = 10).

| | | ESB | EBT | KV | SB |
|---|---|---|---|---|---|
| Preparation | Control | (100) | (100) | (50) | (50) |
| Control | 60.1 | 59.4 | 60.5 | 58.7 | 62.3 |
| ASA 200 + S-NH4 100 | 37.3* | 33.0*^{Λ} | 32.8*^{Λ} | 30.0*^{Λ} | 31.4*^{Λ} |
| IM 10 + FUM 100 | 15.0* | 12.1*^{Λ} | 11.6*^{Λ} | 12.7*^{Λ} | 11.2*^{Λ} |
| NP 100 + VIT C 50 | 25.2* | 20.7*^{Λ} | 21.0*^{Λ} | 20.5*^{Λ} | 22.0*^{Λ} |
| VL 10 + VIT B6 10 | 20.2* | 16.3*^{Λ} | 15.8*^{Λ} | 16.2*^{Λ} | 15.0*^{Λ} |
| BN 60 + GLU 100 | 28.4* | 22.0*^{Λ} | 21.9*^{Λ} | 22.8*^{Λ} | 23.6*^{Λ} |
| PK8 + GLI 100 | 24.3* | 19.3*^{Λ} | 18.2*^{Λ} | 17.9*^{Λ} | 18.0*^{Λ} |

As can be seen from Tables 2-5, polyphenolic compositions and vegetable extracts which contain them, carboxylic acids, amino acids, vitamins increase anti—inflammatory activity of NAIA. Table 6 contains data which demonstrate increase of anti-inflammatory activity of composition which contains NAIA and vitamin, or carboxylic acid, or amino acid.

### EXAMPLE 2

Evaluation of Influence of Carbohydrates and Substances which Possess Sorbing Properties on Toxicity ASA.

Tests were conducted on breedless mice-male with mass 22-26 g. Acute toxicity of ASA was evaluated in accordance with the value LD₅₀ (dose from which 50% of animals perish) of composition containing ACA plus carbohydrate or sorbent with ratio 4:1 when compared with LD₅₀ ASA. Preparations were introduced one time intostomach in form of aqueous suspension with doses 350-3000 mg/kg to groups of six mice. Observations were conducted during 14 days. As a substance which has absorbing properties there were used activated carbon, white clay (caolin), aluminum hydroxide, magnium tricilicate, hydroxylapatite, microcrystaalline cellulose (MCC). As carbohydrates there were used monosaccarides-glucose and fructose, disaccaride-saccarose and lactose, and polysaccaride-potato starch. Results of experiments were processed in accordance with the method of Lichfield-Wilkoxon (Belensky, M. L. “Elements of quantitative evaluation of pharmacological effect,” second edition, redone and added, L, Medgiz, 1963, 152 p).

LD₅₀ composition were counted on ASA (tables 7, 8).

TABLE 7
Influence of substances possessing absorbing activity on
acute toxicity ASA (n = 6)

| | LD₅₀ | |
|---|---|---|
| | mg/kg | Increase of |
| Preparation | ASA | Toxicity In |
| ASA | 2340 | — |
| ASA + activated carbon | 2612 | 1.12 |
| ASA + caolin | 2756 | 1.18 |
| ASA + aluminum | 2695 | 1.15 |
| hydroxide | | |
| ASA + magnium | 2574 | 1.10 |
| tricillicate | | |
| ASA + hydroxy lapitite | 2638 | 1.13 |
| ASA + MCC | 2682 | 1.15 |
| ASA | 2340 | 0 |
| ASA + glucose | 2542 | 1.09 |
| ASA + fractose | 2620 | 1.12 |
| ASA + saccharose | 2530 | 1.15 |
| ASA + lactose | 2632 | 1.08 |
| ASA + starch potato | 2816 | 1.20 |

Judging by the data obtained, substances which possess absorbing properties and also carbohydrates during joint use with NAIA, acetyl salicylic acid reduce its toxicity.

### EXAMPLE 3

Evaluation of influence of carbohydrates and substances possessing sorbing properties in doses 100 mg/kg on pharmacological activity of NAIA in combination with a compound which increases its action, selected from the group: double base carboxylic acids, their salts, amino acids, polyphenolic compounds, products containing them of animal and vegetable origin, vitamins. Doses of used compounds are presented in the table and expressed in mg/kg. Tests were performed on breedless white mice-males with mass 22-26 g.

TABLE 9
Influence of sorbents and carbohydrates on anti-inflammatory activity of
combination of NAIA with carboxylic acids, amino acids, vitamins,
polyphenolic compounds during development of swelling of paw of mice after
introduction of carragenin (N = 10).

| Preparation | Control | AC | TC | MCC | GLU | LAK | FRU |
|---|---|---|---|---|---|---|---|
| Control | 60.1 | 62.2 | 58.3 | 61.7 | 63.0 | 58.8 | 61.1 |
| ASA 200 + AK-NH4 100 | 37.3* | 35.5*^{Λ} | 36.0*^{Λ} | 34.7*^{Λ} | 35.1*^{Λ} | 36.2*^{Λ} | 35.4*^{Λ} |
| IM 10 + FUM 100 | 15.0* | 13.6*^{Λ} | 12.9 | 12.6*^{Λ} | 12.8*^{Λ} | 14.1* | 13.5* |
| NP 100 + VIT C 50 | 26.2* | 23.0*^{Λ} | 22.3*^{Λ} | 24.0*^{Λ} | 22.9*^{Λ} | 22.2*^{Λ} | 22.0*^{Λ} |
| VL 10 + VIT B₆ 10 | 20.2* | 18.4*^{Λ} | 17.8*^{Λ} | 16.9*^{Λ} | 17.7*^{Λ} | 18.5*^{Λ} | 19.0*^{Λ} |
| BN 60 + GLU 100 | 28.4* | 26.3*^{Λ} | 27.0*^{Λ} | 25.9*^{Λ} | 26.7*^{Λ} | 25.2*^{Λ} | 26.4*^{Λ} |
| PK8 + GLy 100 | 24.3* | 20.5*^{Λ} | 21.2*^{Λ} | 22.0*^{Λ} | 22.5*^{Λ} | 21.6*^{Λ} | 22.9*^{Λ} |
| ASA (200) + ESB (150) | 37.3* | 34.2*^{Λ} | 35.0*^{Λ} | 33.1*^{Λ} | 34.9*^{Λ} | 35.3*^{Λ} | 34.7*^{Λ} |
| IM (10) + EBT (100) | 15.2* | 13.1*^{Λ} | 13.7*^{Λ} | 12.6*^{Λ} | 13.2*^{Λ} | 13.0*^{Λ} | 12.0*^{Λ} |
| NP(100) + KV(50) | 23.1* | 20.2*^{Λ} | 21.0*^{Λ} | 19.6*^{Λ} | 21.4*^{Λ} | 20.5*^{Λ} | 20.7*^{Λ} |
| VL (10) + SB (50) | 19.3* | 16.9*^{Λ} | 17.2*^{Λ} | 17.4*^{Λ} | 16.6*^{Λ} | 17.0*^{Λ} | 17.8*^{Λ} |

Note: AC activated carbon, TC-magnesium tricilicate, MCC-monocrystalline cellulose, GLU-glucose, LAC lactose, FRU fructose.

The performed investigation proves an increase of pharmacological activity of investigated combinations of NAIA with carboxylic acids, aminoacids, vitamins, polyphenolic compounds and vegetable extracts containing them, under the action of carbohydrates and substances possessing sorbing properties.

### EXAMPLE 4

Clinical investigation of influence of carbohydrates and substances possessing sorbing properties on exhibiting of side effects of NAIA and their combination with vitamins, carboxylic acids, flavonoids or vegetable extract containing them. Investigations are conducted on 14 ambulatory patients with 32-68 years, both sexes, taking NAIA in form of a course treatment (4-5 weeks) because of acute condition of chronic inflammatory sicknesses; 10 patients were taking acetyl salicylic acid in combination with succinate ammonium for reduction of coagulation of blood after infarction myocarditis. Succinate of ammonium was included in combination for restoration of energy exchange of miyocard. A part of patients (6 people) were taking ortofen (0.05 g) together with vitamins R and C (0.1 g) because of acute condition of rheumatoid arthritis (3 times a day 5 weeks). Preparations were given in form of powder. Three of investigated patients were receiving a mixture of preparations with potato starch (1:1). All patients receiving a combination with starch were taking medication better. They had less exhibited dispeptic phenomenon, headaches, dizziness. Group of 8 men were receiving indometacin together with flavonoid-containing preparation “karsil” because of gout (2 people), thrombofleBeitis (4 people) and rheumatoidal arthritis (2 people). As in the preceding case, preparation was taken as a powder (0.05 g endomethatcyne and 0.25 g karsil 3 times a day first week, and then 4 weeks 4 times a day). Half of the group were receiving preparation together with microcrystalline cellulose in weight ratio 1:2. All patients without exception which received together with combined preparation NAIA and bioflavanoid sorbent of MKT, had side effects of indometnacyne to a lesser degree, in particular dispeptic phenomena, dizziness, headaches, while in the group which did not receive sorbent, 1 patient was forced after the third week to stop taking preparation because of strong headaches in epigastric, vomiting and one more patient needed antyhistamine substances for reduction of allergenic phenomena.

Half of patients of 10 people received acetylsalicylic acid as an antiaggregating substance together with succinate of ammonium (0.25 g and 0.1 g correspondingly), and the second half received the above mentioned combination with activated carbon (0.35 g) two times a day for 8 weeks. Four from five people who received the sorbent exhibited a better taking of mixture ASA and ammonium succinate (they were receiving this combination before), and all 5 exhibited dyspeptic phenomena to a lesser degree. Therefore the obtained data prove a reduction of side effects in combined use of NAIA in combination with other biologically active compounds and carbohydrates or substances possessing sorbing properties.

### EXAMPLE 5

Examples of rational combinations of NAIA with substances which increase pharmacological activity, and also sorbents or carbohydrates are presented. These combinations were obtained after conducting of investigations in experimental way.

Prescription 1

| | |
|---|---|
| Acetylsalicylic acid | 0.5 g |
| Vitamin C | 0.1 g |
| Glucose | up to 0.5 g |

(This prescription is technologically favorable for manufacture).

Prescription 2

| | |
|---|---|
| Acetyl salicylic acid | 0.5 g |
| Rutin | 0.1 g |
| Starch | 0.01 g |
| Glucose | Up to 0.5 g |

(this prescription is technologically favorable for manufacture and highly efficient)

Prescription 3

| | |
|---|---|
| Indometacyn | from 0.025 to 0.05 g |
| Silimarin | 0.01 g |
| Activated carbon | from 0.02 to 0.5 g |

(preparation in all combinations retained high efficiency and low toxicity).

Prescription 4

| | |
|---|---|
| Acetylsalicylic acid | 0.25 g |
| Succinate Ammonium | 0.1 g |
| Extract of badan | 0.1 g |
| KMT | 0.05 g |

Prescription 5

| | |
|---|---|
| Acetylsalicylic acid | 0.4 g |
| Succinate Ammonium | 0.1 g |
| Extract of Badan | 0.03 g |
| Ascorbic acid | 0.03 g |
| Glucose | 0.04 g |

Prescription 6

| | |
|---|---|
| Acetylsalicylic acid | 0.25 g |
| Succinate Ammonium | 0.15 g |
| Extract of malt | 0.05 g |
| Ascorbic acid | 0.05 g |
| Glucose | 0.1 g |

Prescription 7

| | |
|---|---|
| Ortofen | 0.05 g |
| Vitamin R | 0.1 g |
| Vitamin C | 0.1 g |
| Starch | 0.25 g |

Prescription 8

| | |
|---|---|
| Butadion | 0.05 g |
| Glutaminic acid | 0.1 g |
| Lactose | 0.2 g |
| Starch | 0.1 g |
| Magnesium stearate | 0.05 g |

Industrial Applicability

The claimed pharmaceutical compositions can find broad application for treating various sicknesses which require the use of NAIA.

The proposed pharmaceutical composition of various combinations of components which increase pharmacological action of NAIA and at the same time reduce their toxicity and side effects allows to increase efficiency of treatment of patients and reduce the number of negative actions and complications in patients who need to use NAIA.

1. Pharmaceutical composition based on a non-steroid anti-inflammatory agent, containing a non-steroid anti-inflammatory agent and a compound for increasing its pharmaceutical action, reducing toxicity and side effects, characterized in that, as said compound at least one compound from the group including double-base carboxylic acids or their salts, aminoacids or their salts, polyphenolic compounds or products containing them of animal and/or vegetable origin, substances possessing vitaminic action and also additionally at least one special additive which is carbohydrate and/or sorbent.

2. Pharmaceutical composition of claim 1, characterized in that as the non-steroid anti-inflammatory compound it contains acetylsalicylic acid.

3. Pharmaceutical composition of claim 1, characterized in the as products of vegetable origin which contain polyphenolic compositions, it contains extract of badan thick-leaf and/or extract of malt bare.

4. Pharmaceutical composition of claim 1, characterized in that as a substance having vitaminic action it contains ascorbic acid.

5. Pharmaceutical composition of claim 1, characterized in that as the special additive it contains glucose.

6. Pharmaceutical composition in accordance with any claims,
characterized in that it contains in mass %:
acetylsalicylic acid 25-80,
succanate ammonium 5-30,
extract of badan 5-20,
ascorbic acid 5-20,
glucose-the rest.

7. Pharmaceutical composition of claim 1, characterized in that as the carboxylic acid it contains succinic acid.

8. Pharmaceutical composition in accordance with any of the claims, characterized in that it additionally contains a pharmaceutically acceptable carrier which is suitable for oral administration.

9. Pharmaceutical composition in accordance with any of the preceding claims, characterized in that it is formed as a pill or a capsule.
